(19) United States
(12) Patent Application Publication
(10) Pub. No.: US 2001/0033872 A1
(43) Pub. Date: Oct. 25, 2001

(54) Combinations of tyrosine, methylating agents, phospholipids, fatty acids, and St. John's Wort for the treatment of mental disturbances

(76) Inventors: Barbara E. Corson, Fawn Grove, PA (US); Todd R. Henderson, Jarrettsville, MD (US)

Correspondence Address: Covington & Burling, Patent Docketing, 1201 Pennsylvania Avenue NW, Washington, DC, 20004-2401, US

(21) Appl. No.: 09/862,589

(22) Filed: May 23, 2001

Related U.S. Application Data
(63) Continuation of application No. 09/237,222, filed on Jan. 26, 1999, now abandoned.
(60) Provisional application No. 60/072,721, filed on Jan. 27, 1998.
(60) Provisional application No. 60/075,998, filed on Feb. 26, 1998.
(60) Provisional application No. 60/112,993, filed on Dec. 18, 1998.

(51) Int. Cl. A61K 35/78; A61K 31/726; A61K 31/7105; A61K 31/685; A61K 31/525

(52) U.S. Cl. 424/730; 514/78; 514/54; 514/251; 514/561; 514/46

(57) ABSTRACT

This invention provides therapeutic compositions for the treatment or prevention of mental disturbances such as depressive states and for regulating the level of certain neurotransmitters and thereby improving the function of the central nervous system and cognitive function in humans and other animals. The therapeutic compositions comprise any two or more of tyrosine, one or more methylating agents, one or more phospholipids, one or more fatty acids and St. John's Wort (Hypericum perforatum), whether naturally, synthetically, or semi-synthetically derived. The invention also provides a method of administering these compositions to humans or animals in need thereof.

This application is a continuation of U.S. Ser. No. 09/237,222, filed Jan. 26, 1999 and priority is claimed to the following provisional applications: COMPOSITIONS AND METHOD FOR THE TREATMENT OF MENTAL DISTURBANCES, U.S. Ser. No. 60/072,721, filed Jan. 27, 1998; SAMe and OTHER METHYLATING AGENTS, SPHINGOLIPIDS, TYROSINE, ST. JOHN'S WORT COMBINATIONS FOR THE TREATMENT OF MENTAL DISTURBANCES, U.S. Ser. No. 60/075,998, filed Feb. 26, 1998; and SAMe and OTHER METHYLATING AGENTS, PHOSPHOLIPIDS, TYROSINE, ST. JOHN'S WORT COMBINATIONS FOR THE TREATMENT OF MENTAL DISTURBANCES, U.S. Ser. No. 60/112,993, filed Dec. 18, 1998.

### FIELD OF THE INVENTION

The present invention relates to the field of neurohormonal, cognitive and antidepressive therapy. More particularly, the invention pertains to combinations of two or more compounds selected from the group consisting of tyrosine, one or more methylating agents, one or more phospholipids, one or more fatty acids, and St. John's Wort (Hypericum perforatum, whether naturally, synthetically, or semi-synthetically derived, for use in such therapy.

### BACKGROUND OF THE INVENTION

In the mammalian nervous system, nerve impulses are carried across synapses by chemical mediators called neurotransmitters. These compounds are synthesized and stored in the neuron itself, and released into the synaptic cleft. After binding to the post-synaptic neuron, the neurotransmitter is rapidly destroyed or removed, thus terminating its effect. By controlling the synthesis, storage, release, and destruction of neurotransmitters, the body is normally able to control nervous system function. Conversely, derangements in neurotransmitter function cause abnormalities in the functioning of the nervous system. Defective neurotransmission can result from problems with axonal transport, neuronal membrane permeability, lack of precursor substances, or with biosynthesis, release, or inactivation of neurotransmitters. The causes of such derangements of normal neural physiology are as yet incompletely understood. Guyton, A. and Hall, J., Textbook of Medical Physiology, 9^{th} ed., W B Saunders, 1996, pp. 565-82. It is becoming increasingly clear, however, that the manipulation of neuronal processes can serve as a tool for controlling nervous system function. Stein, M., et al., In Vivo and In Vitro Modulation of Central Type Benzodiazepine Receptors by Phosphatidylserine, Molecular Brain Research 5 (1989), pp. 9-15.

Disturbances of mood are increasingly common in western civilization. Although these disturbances have previously been regarded as a sign of “weakness,” science is now aware that these disorders are indeed caused by imbalances in the functions of neurons and the neurotransmitters that they synthesize. At present, several classes of pharmaceuticals are used to treat depression. Serotonin is sometimes called the “feel-good” neurotransmitter: levels of serotonin correlate closely with mood. Increasing serotonin levels in the synaptic cleft results in mood elevation. The selective serotonin reuptake inhibitor pharmaceuticals (“SSRIs”) slow down the process by which serotonin is inactivated (FIG. 1), and thereby increase the level of serotonin within the synaptic cleft. The monoamine oxidase inhibitors (“MOA inhibitors”) inhibit one of the two steps in the enzymatic catabolism of serotonin and the catecholamines (dopamine, epinephrine and norepinephrine) (FIG. 1). Several forms of depression and other CNS diseases (e.g., Parkinson's disease) have been associated with low levels of these neurotransmitters. This inhibition results in an increased level of these neurotransmitters within the synaptic cleft.

Although the SSRIs and the MOA inhibitors have proved of great benefit in the treatment of mental disturbances, both have side effects that preclude their use by some individuals. Side effects commonly seen include anxiety, insomnia, allergic reactions [fluoxetine HCl (tradename, “Prozac”)], and autonomic nervous system disorders including impotence, somnolence, diarrhea, and nausea [sertraline HCl (tradename, “Zoloft”)]. Physicians Desk Reference, Medical Economics Co., Inc., Montvale, N.J., 1999, pp. 924-28, 2443-48.

Aging is often attended by a decrease in cognitive abilities. As the population ages, the need to address this problem without producing harmful side effects will increase. Normal cognition requires many of the same neurotransmitters and biomechanisms that are involved in mood regulation, such as serotonin and the catecholamines. Therefore, therapies that address normalization of neurotransmission (that is, such therapies that would enhance the structure and function of the cell membrane, the production and release of neurotransmitters, and the well-regulated re-uptake or degradation of these neurotransmitters) will improve cognitive CNS function as well as mood. Although, as discussed above, pharmaceuticals exist for the correction of mood disorders, less attention historically has been directed towards the improvement of cognition. Products such as preparations of Ginkgo biloba are available, which are theorized to affect blood supply to the brain, but which have not been shown to address other disorders.

Many age-related neurochemical changes can be traced to structural and functional alterations in neuronal membranes. Specifically, the amount of phosphatidylcholine and phosphatidylethanolamine in the cell membrane decreases with age. These membrane changes, in turn, have been related to changes in phospholipid content in the aging brain. Exogenous administration of phospholipids such as phosphatidylserine may prevent or reverse age-related neurochemical deficits, and has been used to treat such conditions. Champ, P. and Harvey, R., Biochemistry, 2^{nd} ed., Lippincott, Philadelphia, 1994, pp. 266-7; Calderini, G., et al., Phospholipids as Pharmacological Tools in the Aging Brain, Phospholipids in the Nervous System, Physiological Roles, vol. 2: EdL L A Horroacks, Raven Press, New York, N.Y. 1985, p. 11; Bruni, A., et al., Serine Phospholipids in Cell Communication, Phospholipid Research and the Nervous System, L A Horrocks, eds., Laviana Press, Padova, 1986, p. 216; Sun, A. and Sun, G., Neurochemical Aspects of the Membrane Hypothesis of Aging, Interdiscipl. Topics Geront., vol. 15, Karfer, Basel, 1979, pp. 34-53; Pedata, F., et al., Phosphatidylserine Increases Acetylcholine Release from Cortical Slices in Aged Rats, Neurobiology of Aging, vol. 6, 1985, pp. 337-39; Crook, T., et al., Effects of Phosphatidylserine in Age-Associated Memory Impairment, Neurology 41, 1991, p. 644.

Prior art treatments are also limited in their effectiveness because they typically address only one element of CNS function, such as serotonin re-uptake or cerebral blood supply. A composition with multiple mechanisms of action would be an improvement over such prior art treatments because CNS function could be maximally enhanced while doses are minimized. For example, elevation of the serotonin levels in the synaptic cleft will increase the likelihood of serotonin/receptor binding to a certain extent. Cell membrane changes, however, may limit the activity of the receptor. The effects of increasing serotonin levels therefore may be enhanced or even optimized by also improving cell membrane and receptor function. Minimizing doses will also decrease the likelihood of adverse reactions or side effects. There is one currently-available pharmaceutical product (tradename, “Sinemet,” by DuPont) that functions to increase the dopamine levels through two different mechanisms of action. The product contains levadopa, a precursor of dopamine, and carbidopa, which slows the breakdown of dopamine. This product has no effect, however, on membrane fluidity, nor does it enhance the binding of dopamine with receptors. There is a need in the art, therefore, for a composition that combines multiple mechanisms of action to increase levels of dopamine and serotonin, facilitate binding of the neurotransmitters with neuronal cell membranes, and prevent premature deactivation of the neurotransmitters in the CNS, and at the same time contains ingredients having a wide margin of safety and reduced or minimal side effects.

### SUMMARY OF THE INVENTION

It is a primary object of the invention to provide therapeutic and prophylactic compositions that will increase levels of dopamine and serotonin in those individuals suffering from a deficiency of these neurotransmitters, and also improve neuronal cell membrane fluidity, thus enhancing receptor/ligand binding.

It is a further object of the present invention to provide such compositions which result in a low incidence of side effects.

It is a still further object of the present invention to provide a method of regulating and/or improving the levels and function of certain neurotransmitters by administering the compositions of the present invention to humans or other animals in need thereof.

The present invention provides novel compositions and methods for regulating the level of certain neurotransmitters and thereby improving the function of the central nervous system in humans or other animals in need thereof. The compositions of the invention comprise two or more compounds selected from the group consisting of tyrosine, methylating agents, phospholipids, fatty acids and St. John's Wort (Hypericum perforatum), whether naturally, synthetically, or semi-synthetically derived, for use in the facilitation of improved neurotransmitter levels and function in humans or animals. Combinations containing methylating agents, phospholipids and/or fatty acids may contain one or more of each such class of compounds.

### BRIEF DESCRIPTION OF THE DRAWINGS

FIG. 1 is a sequence for the degradation of dopamine.

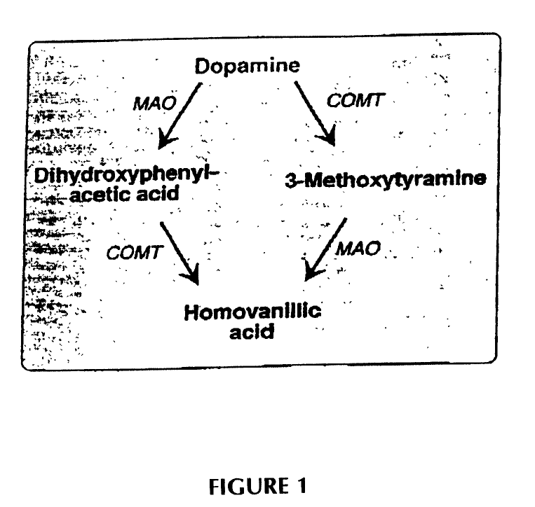

FIG. 2 is the molecular structure of tyrosine.

FIG. 3 is a sequence for the biosynthesis of catecholamines from tyrosine.

FIG. 4 is the molecular structure of S-adenosylmethionine.

FIG. 5 is the molecular structure of 5-methyltetrahydrofolate.

FIG. 6 shows the synthesis of phosphatidylcholine from phosphatidylserine.

### DETAILED DESCRIPTION OF THE INVENTION

In accordance with the teachings of the present invention, disclosed herein are therapeutic and/or prophylactic compositions capable of increasing the levels and function of certain neurotransmitters in the mammalian CNS and thereby treating or preventing certain diseases associated with or resulting in deficiencies in levels or function of those neurotransmitters and improving the structure and function of the mammalian CNS. The invention relates to novel compositions comprising two or more compounds selected from the group consisting of tyrosine, one or more methylating agents, one or more phospholipids, one or more fatty acids and extracts of Hypericum perforatum (St. John's Wort), whether naturally, synthetically or semi-synthetically derived.

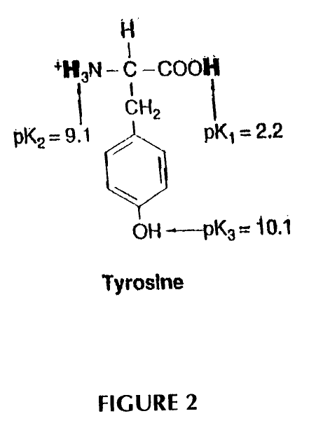

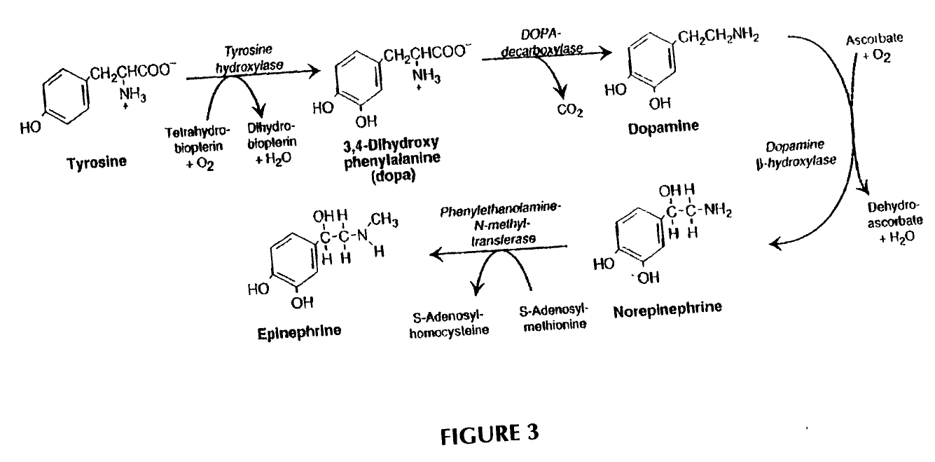

The amino acid tyrosine (FIG. 2) is important to central nervous system (“CNS”) function because it is a precursor of the neurotransmitters dopamine, norepinephrine and epinephrine (FIG. 3). Champ and Harvey, Biochemistry, 1994. It is well established that tyrosine intake can directly affect levels of these neurotransmitters. Tyrosine is also a constituent of the enkephalins and endorphins, which function as natural painkillers and mood regulators. Tyrosine increases the levels of these neurotransmitters in the body. Braverman, E., Tyrosine, The Healing Nutrients Within, Keats Publishing, New Canaan, Conn., p. 44; Costanzo, L., Physiology, Williams and Wilkins, 1995, p. 16; Jahng, J., et al., Differential Expression of Monoamine Oxidase A, Serotonin Transporter, Tyrosine Hydrosylase and Norepinephrine Transporter mRNA by Anorexia Mutation and Food Deprivation, Brain Res. Dev. 107(2), 1998, pp. 241-46; Champ and Harvey, Biochemistry, 1994. Tyrosine is indicated in diseases associated with a deficit of these neurotransmitters, including Parkinson's disease and attention deficit disorder.

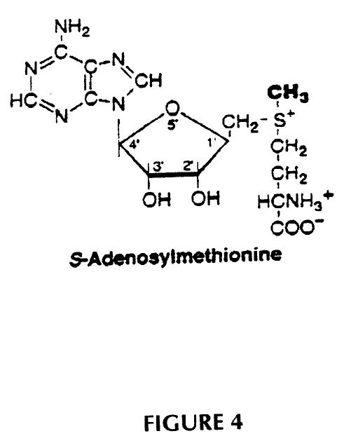

Methylating agents are essential for numerous reactions in the brain including maintaining cell membranes and synthesis of neurotransmitters. Decrease in levels of the methylating agents S-adenosylmethionine (“SAMe”) and 5-methyltetrahydrofolate (“5-MTHF”) has been associated with age-related neurologic deficits and numerous psychiatric disorders, particularly depressive states. SAMe is a compound synthesized in the body from adenosine triphosphate (“ATP”) and methionine (FIG. 4). It is present in many tissues, including the central nervous system. The primary CNS function of SAMe is to donate methyl groups in the reactions synthesizing various crucial compounds, including neurotransmitters and phospholipids. For example, SAMe facilitates the conversion of phosphatidylethanolamine to phosphatidylcholine, which forms part of the inner, lipid layer of the plasma membrane. In so doing, SAMe increases membrane fluidity and enhances effectiveness of receptor/ligand binding. (Champ and Harvey, Biochemistry, 1994; Stramentinoli, G., Pharmacologic Aspects of S-Adenosylmethionine, American Journal of Medicine 83 (5A), 1987, p. 35; Baldessarini, F., Neuropharmacology of S-Adenosyl Methionine, American Journal of Medicine 83 (5A), 1987, p. 95; Carney, M., Neuropharmacology of S-Adenosyl Methionine, Clinical Neuropharmacology 9(3), 1986, p. 235; Janicak, P., S-Adenosylmethionine in Depression, Alabama Journal of Medical Sciences 25(3), 1988, p. 306. These functions may also pertain to other methyl donors such as betaine (trimethylglycine), 5-methyltetrahydrofolate, folic acid, and dimethylglycine. Champ and Harvey, Biochemistry, 1994.

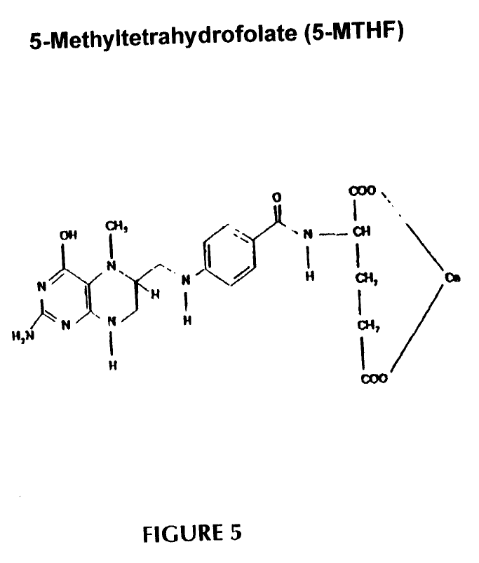

The methylating agent 5-methyltetrahydrofolate (“5-MTHF”) (FIG. 5), which is the active form of folate, has advantages over some other methylating agents, such as folic acid, because it can cross the blood-brain barrier. Unlike folic acid, 5-MTHF does not mask vitamin B-12 deficiency (pernicious anemia). It is absorbed and utilized in the body without being transformed. Moreover, 5-MTHF has the important role of converting homocysteine into methionine. Hyperhomocysteinemia has been implicated in hypercholesterolemia and vascular diseases, and 5-MTHF is a valuable compound for the treatment of these significant conditions. Champ and Harvey, Biochemistry, 1994; Abou-Saleh, M., The Biology of Folate in Depression: Implications for Nutritional Hypothesis of the Psychoses, J. Psychiatr. Res. 20(2), 1986, pp. 91-101; Matthews, R., et al., Methylenetetrahydrofolate Reductase and Methionine Synthase: Biochemistry and Molecular Biology, Eur. J. Pediatr. 157(2), 1998, pp. S54-59; Stramentinoli G., General Information on 5-MTHF, Knoll Farmaceutici Spa, unpublished material which may be obtained from the author at Via Fosse Ardeatine, 2-1-20060 Liscate (Mi); Loehrer, F., et al., Influence of Oral S-Adenosylmethionine on Plasma 5-Methyltetrahydrofolate, S-Adenosylhomocysteine, Homocysteine and Methionine in Healthy Humans, J. Pharmacol. Exp. Ther. 282(2), 1997, pp. 845-50; Verhaar, M., 5-Methyltetrahydrofolate, the Active From of Folic Acid, Restores Endothelial Function in Familial Hypercholesterolemia, Circulation 27(3), 1998, pp. 237-41.

It is expected that any methylating agent, such as, but not limited to, SAMe, 5-MTHF, folate, betaine (dimethylglycine), and trimethylglycine, can be included in the compositions of the present invention. The preferred methylating agents in the compositions of the present invention are SAMe, 5-MTHF and betaine. These preferred methylating agents may be included in the compositions of the present invention either individually or in mixtures with themselves or with other methylating agents.

The phospholipids are a class of lipid molecules of particular importance in the function of the mammalian cell. They make up 75% of cell membranes, and therefore play an essential role in interactions between cells, including the communications that pass between neurons in the brain. Phospholipids are responsible for maintaining cell membrane fluidity. In other words, phospholipids allow cell receptors to be mobile over the surface of the cell, thereby enhancing cell-to-cell communication. This is important in all tissues, but especially so in the CNS. Phospholipids increase the effectiveness of existing levels of neurotransmitters, by ensuring that a maximum number of neurotransmitter molecules are able to interface with a receptor molecule.

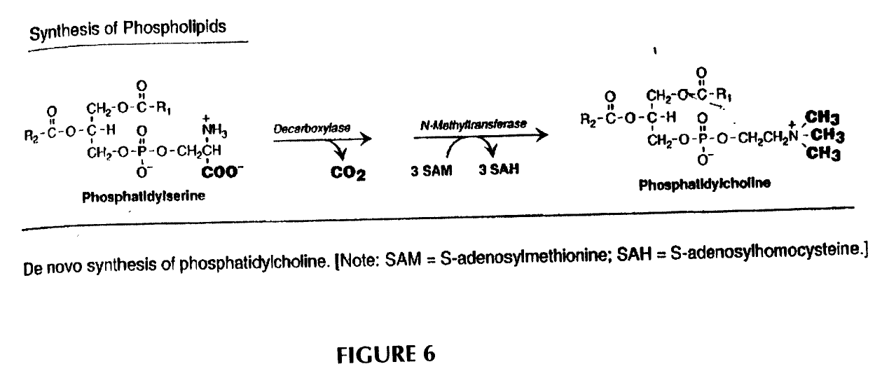

There are two general classes of phospholipids, namely, phosphoglycerides and sphingolipids. The two groups of phospholipids are interrelated in both structure and function. Both are amphipathic, that is, they have a hydrophilic head composed of a phosphate group and a long hydrophobic tail composed of one or more fatty acid chains. Attached to the phosphate head are various moieties that characterize the different phospholipids. It is the amphipathic nature of phospholipids that enables them to function in the cell membrane. Examples of phosphoglycerides that are important to CNS structure and function include phosphatidylserine, phosphatidylcholine, phosphatidylethanolamine, phosphatidylglycerol, phosphatidylinositol, and cardiolipin. Phosphatidylserine is the precursor of phosphatidylcholine (FIG. 6), which is the single most abundant phospholipid in mammalian cell membranes. Examples of sphingolipids that are essential to the structure and function of the CNS include sphingomyelin, ceramide, gangliosides, and cerebrosides. The preferred phospholipids in the compositions of the present invention are phosphatidylserine, phosphatidylcholine, and sphingomyelin. These preferred phospholipids may be included in the compositions of the present invention either individually or in mixtures with themselves or with other phospholipids.

Long-chain fatty acids serve as precursors of phospholipids in the body, and may have independent neurochemical effects as well. Exogenously administered fatty acids are combined within the cell with phosphate moieties donated by ATP molecules to form phospholipids. The most important fatty acids in this regard are those over seventeen carbons in length, such as linoleic acid, linolenic acid, oleic acid, stearic acid, arachidonic acid, docosahexaenoic acid, eicosapentaenoic acid, lignoceric acid and nervonic acid. The preferred fatty acids to be included in the compositions of the present invention are arachidonic acid, docosahexaenoic acid, and nervonic acid. These preferred fatty acids may be included in the compositions of the present invention either individually or in mixtures with themselves or with other fatty acids.

St. John's Wort (Hypericum perforatum) (“SJW”) is an herbaceous plant native to Europe and North America. The plant contains several pharmacologically active compounds including hypericin. The exact mechanism of action of SJW is not clear, but infusions of the herb have been documented to inhibit catechol-o-methyl-transferase (“COMT”), and monoamine oxidase (“MAO”) types A and B. These enzymes normally catabolize the catecholamines, including dopamine, serotonin, epinephrine, and norepinephrine. Blocking COMT and MAO A and B results in increased levels of serotonin and other catecholamines. St. John's Wort and hypericin fraction extracts of this herb inhibit MAO and COMT, therefore slowing the rate of breakdown of serotonin, the “feel good” neurotransmitter. This action is advantageous in almost all neurologic conditions because elevation of mood and reduction of perceived stress have beneficial effects on all body systems. In particular, St. John's Wort is indicated in affective disorders, such as depressive states. Its mechanism is similar to that of commonly prescribed MAO-inhibiting pharmaceuticals. Additionally, SJW may block the re-uptake of serotonin in the synaptic cleft, thus prolonging its effect. In this effect, SJW appears to duplicate the effects of SSRIs. Additionally, SJW may also affect the metabolism of other neurotransmitters such as dopamine, GABAA and GABAB. Numerous studies have documented the positive effect of SJW extracts on CNS function. Wincor, M. and Gutierrez, M., St. John 's Wort and the Treatment of Depressions, Pharmacist, August, 1997, p. 88; Cicero, L., et al., Can Depression Patients Be Treated with Worts?, Drug Topics, September, 1997, p. 24; Tyler, V., The Honest Herbal, Haworth Press, Inc., New York, 1993, p. 275; Raffa, R., Screen of Receptor and Uptake-Site Activity of Hypericin Component of St. John's Wort Reveals Sigma Receptor Binding, Life Sci. 62(16), 1998, pp. PL265-70; Cott, J., In Vitro Receptor Binding and Enzyme Inhibition by Hypericum Perforatum Extract, Pharmacopsychiatry, 30(2), 1997: pp. 108-12; Duke, J., et al., Western Herbal Medicine: Traditional Materia Medica, Complementary and Alternative Veterinary Medicine, Schoen, A., Wynn, S., eds., Mosby, St. Louis, Mo. 1998, pp. 329, 357.

Tyrosine, the various phospholipids, SAMe and 5-MTHF are available in pure and stabilized forms for oral dosing. The active compounds in St. John's Wort are not completely characterized but research has centered on hypericin, xanthones, and flavinoids present in the plant leaves and flowers. A standardized extract of the herb containing 0.3% hypericin is available. The dose of this extract for an adult human is typically 300 mg per day. Although hypericin is not the only active compound in the plant, it apparently is a marker for the fraction of the extract that does contain many if not all of the active phytochemicals. Methods of analysis, both in vivo and in vitro, exist for tyrosine, phospholipids, SAMe, 5-MTFH, and hypericin.

Examples of the combinations of the present invention include:

1. Tyrosine and S-adenosylmethionine

2. Tyrosine and 5-methyltetrahydrofolate

3. Tyrosine, phosphatidylserine, and S-adenosylmethionine

4. St. John's Wort and 5-methyltetrahydrofolate

5. S-adenosylmethionine and St. John's Wort

6. Tyrosine and St. John's Wort

7. Tyrosine, S-adenosylmethionine, and St. John's Wort

8. St. John's Wort and phosphatidylserine

9. Phosphatidylcholine, phosphatidylinositol and sphingomyelin

10. Arachidonic acid, SAMe and tyrosine

11. 5-MTHF and docosahexanoic acid

These compounds have different mechanisms of action, but all are crucial parts of biochemical reactions that result in a net increase in the levels of the important neurotransmitters dopamine and serotonin, and in improving the overall structure and function of the central nervous system.

It is expected that elements of the combinations of the present invention will work synergistically together because they have different, but complementary, mechanisms of action. For example, tyrosine will function to increase dopamine levels. The beneficial effects of such an increase would be multiplied if membrane fluidity were also increased, thereby facilitating interactions between the neurotransmitter and the receptor neuron. Administration of a methylating agent, such as SAMe, will increase membrane fluidity. Therefore, a composition combining tyrosine and SAMe can be expected to function synergistically to increase dopamine levels and simultaneously enhance the effect of the dopamine on the target receptor neuron. Similarly, an increase in dopamine levels would be of limited value if the dopamine were quickly broken down by MAO and COMT. St. John's Wort functions to increase net dopamine levels by increasing the half-life of this neurotransmitter. While tyrosine, SAMe or St. John's Wort administration separately theoretically might each be expected to increase neuron function by 5-10%, a combination of two of these compounds theoretically might be expected to increase neuron function by 30-40%. As these examples show, the effects of combinations would be expected to be more than merely additive.

Such synergy also enables the reduction of dosage levels for each compound. For example, the amount of SAMe required to achieve a given improvement in neural function would be lower if the SAMe were administered in combination with tyrosine or St. John's Wort than if it were given in isolation. This reduction in dose correspondingly would reduce the potential for side effects. The compounds included in the composition of the present invention have wide margins of safety and low incidences of side effects in any case. Large doses of tyrosine can be associated with headaches, and overdoses of St. John's Wort have caused cases of photosensitization in humans and animals. At therapeutic levels, however, the incidence of side effects associated with these compounds is very low and certainly less than that of the prior art pharmaceuticals discussed above, such as the SSRIs. By combining compounds and therefore lowering necessary doses, the risk of side effects is even further reduced.

In many incidences of CNS dysfunction, moreover, the exact etiology can only be surmised. The compositions of the present invention are thus also superior to prior art treatments in that they can target numerous elements of CNS function, including cell membrane function (phospholipids, SAMe), vascular function (5-MTHF), neurotransmitter production (tyrosine, SAMe) and prevention of neurotransmitter degradation (St. John's Wort).

The compositions of the present invention can be administered by a variety of routes including, but not limited to: orally, transdermally, sublingually, intravenously, intramuscularly, rectally, and subcutaneously. Preferred daily doses for each of the compounds are as follows:

SAMe (or many other methylating agents)

Total dose range: 15 mg to 5000 mg

Preferred small animal dose range: 15 mg to 600 mg

Preferred human dose range: 25 mg to 1000 mg

Preferred large animal dose range: 500 mg to 5000 mg

Tyrosine

Total dose range: 50 mg to 5000 mg

Preferred small animal dose range: 50 mg to 2500 mg

Preferred human dose range: 100 mg to 2500 mg

Preferred large animal dose range: 500 mg to 5000 mg

St. John's Wort

Total dose range: 50 mg to 5000 mg

Preferred small animal dose range: 50 mg to 500 mg

Preferred human dose range: 1100 mg to 600 mg

Preferred large animal dose range: 500 mg to 5000 mg

Standardized St. John's Wort fraction containing hypericin

Total dose range: 0.25 mg to 500 mg

Preferred small animal dose range: 0.25 mg to 25 mg

Preferred human dose range: 2 mg to 25 mg

Preferred large animal dose range: 25 mg to 500 mg

5-Methyltetrahydrofolate

Total dose range: 10 micrograms to 500 mg

Preferred small animal dose range: 10 micrograms to 200 mg

Preferred human dose range: 20 micrograms to 250 mg

Preferred large animal dose range: 30 micrograms to 500 mg

Phospholipids (example: phosphatidylserine or mixtures of phospholipids)

Total dose range: 5 mg to 5000 mg

Preferred small animal dose range: 5 mg to 500 mg

Preferred human dose range: 10 mg to 3000 mg

Preferred large animal dose range: 500 mg to 5000 mg

Fatty Acids (example: arachidonic acid)

Total dose range: 2.5 mg to 2500 mg

Preferred small animal dose range: 2.5 mg to 250 mg

Preferred human dose range: 5 mg to 1500 mg

Preferred large animal dose range: 250 mg to 2500 mg

Having discussed the composition of the present invention, it will be more clearly perceived and better understood from the following specific examples which are intended to provide examples of the preferred embodiments and do not limit the present invention. Moreover, as stated above, the preferred components described in these examples may be replaced by or supplemented with the any of the components of the compositions of the invention described above.

### EXAMPLE 1

A 72 year old woman has Parkinson's disease. This neurological dysfunction is associated with degeneration of the dopaminergic neurons of the substantia nigra and locus ceruleus of the brain. The symptoms of the disease (tremor, slowness of voluntary movement, facial rigidity and mental confusion) are a result of the decrease in dopamine in the substania nigra. The patient is given a composition comprising 500 mg per day of tyrosine, 200 mg per day of SAMe and 300 mg per day of phosphatidylcholine. The tyrosine functions as a precursor of dopamine, and its administration results in increased dopamine levels. The SAMe and phosphatidylcholine result in enhanced neuronal cell membrane fluidity, thereby allowing the dopamine to be more effectively used. Phosphatidylcholine and SAMe also enhance neuronal health and therefore support the remaining dopaminergic neruons and help maintain their secretion of dopamine.

This treatment for Parkinson's disease is an improvement over prior art therapies. The current treatment of choice, which is the simultaneous administration of levadopa and carbidopa, provides a narrow therapeutic range (the effective dose is close to the dose at which adverse reactions such as dyskinesias, or involuntary movements, occur). Levadopa and carbidopa are also contraindicated in persons with cardiovascular, renal or pulmonary disease, which are common in the elderly. The combination of tyrosine, SAMe and phosphatidylcholine has a much wider therapeutic margin and no known contraindications.

### EXAMPLE 2

A 9 year old neutered male cat has been “spraying” (urine marking) in his owner's house since the adoption of a second cat. The attending veterinarian diagnoses socially related inappropriate elimination, a condition caused by anxiety and mental conflict in the cat. Since this condition is the result of decreased serotonin levels, it can be treated by administering a combination of St. John's Wort and phospholipids. The patient is given a composition comprising 100 mg per day of St. John's Wort containing 0.3% hypericin, and 100 mg per day of phosphatidylserine. The effect of the St. John's Wort is to increase the half-life of serotonin, thereby increasing its potential to affect a target neuron. The phosphatidylserine increases neuronal cell membrane fluidity and maximize the binding of serotonin to the post-synaptic receptors, further increasing this desirable effect and allowing the dosage of St. John's Wort to be minimized.

This is an improvement over current therapy which consists of administering pharmaceuticals such as diazepam (a benzodiazapine), amitriptyline (a tricyclic antidepressant), megesterol acetate (a synthetic progesterone) or fluoxetine (a SSRI). These pharmaceuticals are associated with side effects and some are controlled substances with abuse potential. Moreover, none is 100% effective, and there exists a need to develop additional alternatives to treat those animals that do not respond to these pharmaceuticals

### EXAMPLE 3

A 65 year old man is recovering from an episode of cerebral hypoxia due to ischemia and infarction, commonly called a stroke. In this injury to the brain, an interruption in the supply of oxygen fatally injures brain cells (neurons). The resulting death of neurons causes deficits in neural function and leads to varying symptoms including paralysis, amnesia, and aphasia (loss of speech). Although the destroyed neurons cannot be replaced, alternative pathways may develop that allow lost skills (walking, speech) to be relearned. Repairing the damaged CNS tissue and developing these alternative pathways requires the activity of non-neuronal cells in the CNS, which are collectively called glial cells. The administration of SAMe and phospholipids would be very beneficial in the aftermath of cerebral injury. The patient is therefore given a composition comprising 400 mg per day of SAMe and 450 mg per day of mixed phospholipids (250 mg of phosphatidylcholine, 100 mg of phosphatidylserine and 100 mg of sphingomyelin). The phosphatidylserine is administered in this case because phospholipids make up 75% of the cell membrane and are therefore necessary for growth and function of glial cells, including astrocytes and microglial cells. SAMe is an essential participant in synthesis reactions in healing and growing CNS tissue. For example, it participates in the formation of phosphatidylcholine (the single most abundant phospholipid in cell membranes) from phosphatidylserine.

### EXAMPLE 4

A 58 year old man has seasonal affective disorder, in which the decreasing hours of natural daylight in fall and winter months have a depressant effect on mood. This condition is associated with decreased amounts of serotonin. Current therapy for this condition includes the SSRI's, MAO inhibitors or administration of St. John's Wort alone. Because these approaches utilize the same mechanism of action, i.e., they all slow the rate of serotonin degradation by blocking the action of COMT and/or MAO, they have additive effects in that they prolong the half life of serotonin. Therefore, using more than one of these compounds simultaneously can cause adverse effects by delaying the breakdown of serotonin too long, leading to nausea, vomiting, mental confusion and involuntary eye movements. Using MAO inhibitors and St. John's Wort together would not offer additional improvement in neural function, because the limiting factor of serotonin receptors on the post-synaptic cell is not addressed by either compound. By administering St. John's Wort in combination with phospholipids, however, serotonin levels can be elevated while receptor accessibility on the post-synaptic cell is also increased, thus insuring that almost every molecule of serotonin is used to good effect. The patient is therefore given a composition comprising 100 mg per day of St. John's Wort containing 0.3% hypericin and 150 mg per day of phosphatidylserine.

### EXAMPLE 5

A 7 year old Standardbred gelding has developed signs of ataxia and muscle weakness due to infection with Sarcocytis neurona, or Equine Protozoal Myeloencephalitis. This disease is caused by aberrant parasite migration through the descending and ascending nerve pathways in the spinal cord and in the brain. Although the actual infection can apparently be terminated by long term administration of antibiotics, the inventors are aware of no compound available for use in horses that will aid in healing the damage done by the parasitic migration. The disease leaves many horses with severe neurological deficits. The patient is given a composition comprising 800 mg per day of SAMe and 1300 mg per day of neurohomologous phospholipids, including 900 mg per day of phosphatidylserine and 400 mg per day of sphingomyelin. As in Example 3, this composition would aid in the clearing away of damaged tissues and the growth and repair of glial cells that would facilitate the formation of alternative pathways, thereby enhancing recovery.

### EXAMPLE 6

A 48 year old man is concerned because he has increased difficulty with cognitive function, specifically short-term memory and attention span. These symptoms are associated with several pathological changes in the CNS. One of these changes is the decreased neuronal mass that results as neurons succumb over time to toxins such as alcohol, hypoxia due to subclinical ischemia, and age-associated changes in cell membrane phospholipid content. Age associated cognitive dysfunction has also been linked to the brain's decreasing capacity to produce adequate amounts of neurotransmitters like dopamine and norepinephrine. Therefore, this patient is treated with a composition comprising 500 mg per day of tyrosine, 300 mg per day of phospholipids (200 mg per day of phosphatidylserine and 100 mg per day of phosphatidylcholine) and 200 mg per day of SAMe. The tyrosine increases dopamine and norepinephrine levels in the brain. Simultaneously, the phospholipids and SAMe enhance cell membrane function and facilitate neurotransmitter/receptor binding.

Many modifications may be made without departing from the basic spirit of the present invention. Accordingly, it will be appreciated by those skilled in the art that within the scope of the appended claims, the invention may be practiced other than has been specifically described herein. Hence, the attached claims are intended to cover the invention embodied in the claims and substantial equivalents thereto.

We claim:

1. A composition comprising any two or more of the following compounds: tyrosine, one or more methylating agents, one or more phospholipids, one or more fatty acids and St. John's Wort (Hypericum perforatum).

2. The composition of claim 1 in which the daily dose of tyrosine for humans or animals ranges from 50 milligrams to 5000 milligrams.

3. The composition of claim 1 in which the methylating agent or agents is or are selected from the group consisting of S-adenosylmethionine, 5-methyltetrahydrofolate, folate, betaine (dimethylglycine), and trimethylglycine.

4. The composition of claim 3 in which one or more of the methylating agents is S-adenosylmethionine and in which the daily dose for humans or animals ranges from 15 milligrams to 5000 milligrams.

5. The composition of claim 3 in which one or more of the methylating agents is 5-methyltetrahydrofolate and in which a daily dose for humans or animals ranges from 10 micrograms to 500 milligrams.

6. The composition of claim 1 in which the phospholipid or phospholipids is or are selected from the group consisting of phosphatidylserine, phosphatidylcholine, phosphatidylethanolamine, phosphatidylglycerol, phosphatidylinositol, cardiolipin, sphingomyelin, ceramide, gangliosides, and cerebrosides.

7. The composition of claim 6 in which the daily dose of the phospholipid or phospholipids for humans or animals ranges from 5 milligrams to 5000 milligrams.

8. The composition of claim 1 in which the fatty acid or acids is or are selected from the group consisting of all fatty acids over 17 carbons in length.

9. The composition of claim 8 in which the fatty acid or acids is or are selected from the group consisting of linoleic acid, linolenic acid, oleic acid, stearic acid, arachidonic acid, docosahexaenoic acid, eicosapentaenoic acid, lignoceric acid and nervonic acid.

10. The composition of claim 8 in which the daily dose of fatty acid or acids for humans or animals ranges from 2.5 milligrams to 2500 milligrams.

11. The composition of claim 1 in which the daily dose of St. John's Wort for humans or animals ranges from 50 milligrams to 5000 milligrams.

12. The composition of claim 1 in which the daily dose of the standardized extract of St. John's Wort (hypericin fraction) for humans or animals ranges from 0.25 milligrams to 500 milligrams.

13. A method of administering the composition of the present invention to a human or other animal.

14. A method of regulating the neurotransmitters of a human or other animal by administering therapeutically or prophylactically effective amounts of the compositions of the present invention.

15. A method of normalizing or improving the function of the central nervous system of a human or other animal by administering a therapeutically or prophylactically effective amount of the compositions of the present invention.
